(19) United States
(12) Patent Application Publication
(10) Pub. No.: US 2021/0238635 A1
(43) Pub. Date: Aug. 5, 2021

(54) DIGESTER COMPRISING AN OXYGEN INJECTION SYSTEM HAVING A TUBULAR MEANS COMPRISING A T-SHAPED PORTION

(71) Applicant: L'Air Liquide, Société Anonyme pour l'Etude et l'Exploitation des Procédés Georges Claude, Paris (FR)

(72) Inventors: Aude BERTRANDIAS, Paris (FR); David FRIMAT, Paris (FR); Antonio TRUEBA, Charenton le Pont (FR); Jacopo SEIWERT, Versailles (FR); Jeremy OLLIER, Mérignac (FR); Solene VALENTIN, Voreppe (FR)

(21) Appl. No.: 17/144,762

(22) Filed: Jan. 8, 2021

(30) Foreign Application Priority Data
Jan. 9, 2020 (FR) 2000179

(51) Int. Cl. C12P 5/02; C12P 3/00; C12M 1/00; C12M 1/26

(52) U.S. Cl. CPC C12P 5/023; C12M 33/04; C12M 29/00; C12P 3/00

(57) ABSTRACT

A plant for producing at least partially desulfurized biogas, comprising a biomass digester and/or post-digester, the digester and/or post-digester comprising:
a chamber (1) comprising the biomass (2) and the gas space (3), and
a system (6) for injecting an oxidizing gas into the gas space,
   wherein the injection system comprises a tubular means (4) comprising a T-shaped portion the vertical bar of which is placed in the axis of symmetry of the chamber and the horizontal bar of which has micro-injectors (5).

### CROSS REFERENCE TO RELATED APPLICATIONS

This application claims the benefit of priority under 35 U.S.C. § 119 (a) and (b) to French Patent Application No. 2000179, filed Jan. 9, 2020, the entire contents of which are incorporated herein by reference.

### TECHNICAL FIELD

The present invention relates to a plant and a process for producing at least partially desulfurized biogas.

### BACKGROUND

Biogas is the gas produced during the decomposition of organic matter in the absence of oxygen (anaerobic digestion), also known as methanization. The decomposition may be natural, as observed in swamps or in household rubbish dumps, however the production of biogas may also result from the methanization of wastes in a dedicated reactor, under controlled conditions, known as a methanizer or digester, and then in a post-digester, which is similar to the digester and allows the methanization reaction to be extended.

Biomass refers to any group of organic matter that can be converted into energy through this methanization process: for example, treatment plant sludges, manures/liquid manures, agricultural residues, food wastes, etc.

The digester, that is to say the reactor dedicated to the methanization of the biomass, is a closed vessel, heated or not (operated at a set temperature, between the ambient temperature and 55° C.), the contents of said vessel, composed of the biomass, being mixed, continuously or sequentially. The conditions in the digester are anaerobic, and the biogas generated is found in the headspace of the digester (gas space), from where it is withdrawn. Post-digesters are similar to digesters.

Owing to its main constituents—methane and carbon dioxide—biogas is a powerful greenhouse gas; at the same time, it also constitutes a source of renewable energy, which is appreciable in the context of the increasing scarcity of fossil fuels.

Biogas contains predominantly methane (CH₄) and carbon dioxide (CO₂), in proportions which can vary according to the substrate and to the way in which the biogas is obtained; however it may also contain, in smaller proportions, water, nitrogen, hydrogen sulfide (H₂S), oxygen, and also other organic compounds, in the form of traces, including H2S, between 10 and 50 000 ppmv.

Depending on the organic matter which has been decomposed and on the techniques used, the proportions of the components differ but, on average, biogas comprises, on a dry gas basis, from 30% to 75% of methane, from 15% to 60% of CO₂, from 0% to 15% of nitrogen, from 0% to 5% of oxygen and trace compounds.

Biogas is made use of economically in various ways. It can, after a gentle treatment, be exploited close to the production site in order to supply heat, electricity or a mixture of both (cogeneration); the high carbon dioxide content reduces its calorific value, increases the costs of compression and of transportation and limits the economic advantage of making use of it economically to this use nearby.

More intensive purification of biogas allows it to be more widely used; in particular, intensive purification of biogas makes it possible to obtain a biogas which has been purified to the specifications of natural gas and which can be substituted for the latter biogas thus purified is known as “biomethane”. Biomethane thus supplements natural gas resources with a renewable part produced within territories; it can be used for exactly the same uses as natural gas of fossil origin. It may supply a natural gas network or a vehicle filling station; it may also be liquefied for storage in the form of liquefied natural gas (bioLNG), etc.

Depending on the composition of the biomass, the biogas produced during the digestion contains hydrogen sulfide (H₂S) in amounts of between 10 and 50 000 ppm.

Irrespective of the final commercial destination of the biogas, it proves to be vital to remove hydrogen sulfide, which is a toxic and corrosive impurity. Moreover, if the use of the biogas involves purifying it for injection of biomethane into the natural gas network, there are strict specifications limiting the permitted quantity of H₂S.

A number of methods exist for removing H₂S and are more or less widespread (beds of activated carbon, addition of iron compounds, physical or chemical absorption, water washing, biofilters, etc.). Removal is accomplished primarily by adsorption on a bed of activated carbon, outside the digester. In an increasing number of digesters, H₂S reduction is also accomplished in part by injecting air/enriched air/O₂ into the gas space of the digester, this constituting an in situ solution. With injection into the gas space at a low dose, solid sulfur is formed from the H₂S and O₂ (eq. (1)), this being performed by sulfur-oxidizing bacteria, e.g. Thiobacillus. With a high dose of O₂ injected, the mixture is acidified (eq. (2)). The target reaction is therefore reaction (1).

H₂S+0.5O₂→S+H₂O (1)

H₂S+2O₂→SO₄²⁻+2H+ (2)

The amounts of O₂ which need to be injected in practice are different from those expected from the stoichiometry of eq. (1): doses of 0.3%-3% O₂ relative to the biogas generated are most frequently recommended, with doses of up to 12% being sometimes stated.

Presently, the in situ injection of air/enriched air/O₂ is not optimized, and the beds of activated carbon must therefore be maintained in order to remove all of the H₂S.

Existing solutions consist in injecting air/O₂ into the digester using a single injection point, which potentially leads to a localized reaction (localized removal of hydrogen sulfide). In the event of inadequate oxygen dosage, this can furthermore lead to a local accumulation of unconsumed oxygen, which is undesirable since (i) the mixture of biogas and oxygen is explosive beyond a certain limit, (ii) the purification of biogas containing oxygen is more complex, (iii) the accumulated oxygen is not used for reducing H₂S to the maximum extent possible, and (iv) aerobic zones may be created locally and inhibit the methanization reaction.

Moreover, the single injection point is placed opposite the biogas outlet. Part of the biogas may thus be discharged from the digester directly without having had the chance to react with the injected oxygen. It thus remains contaminated with hydrogen sulfide at the outlet.

Specifically, according to simulations of a digester represented in FIG. 1 and FIG. 2, it can be seen that the biogas flows in a laminar mode. This confirms that the oxygen injected at one point is not mixed into the whole of the gas space of the digester, but is carried directly to the gas outlet. The reaction is therefore localized.

This has been confirmed visually since it has been observed that solid sulfur was formed more on the surfaces of the desulfurization net located at the periphery and less so at the centre.

From this basis, one problem which arises is that of providing an improved plant promoting more intensive removal of H₂S.

### SUMMARY

One solution of the present invention is a plant for producing at least partially desulfurized biogas, comprising a biomass digester and/or post-digester, the digester and/or post-digester comprising:
a chamber 1 comprising the biomass 2 and the gas space 3, and
a system 6 for injecting an oxidizing gas into the gas space,
   characterized in that the injection system comprises a tubular means 4 comprising a T-shaped portion the vertical bar of which is placed in the axis of symmetry of the chamber and the horizontal bar of which has micro-injectors 5.

“Gas space” refers to the space in the digester or post-digester that contains gas (as opposed to the space which contains the liquid).

### BRIEF DESCRIPTION OF THE DRAWINGS

For a further understanding of the nature and objects for the present invention, reference should be made to the following detailed description, taken in conjunction with the accompanying drawings, in which like elements are given the same or analogous reference numbers and wherein:

FIG. 1 is a cross-sectional view of the biogas flows in a laminar mode:

FIG. 2 is a perspective view the biogas flows in a laminar mode; and

FIG. 3 is a vertical schematic section through the chamber of the digester.

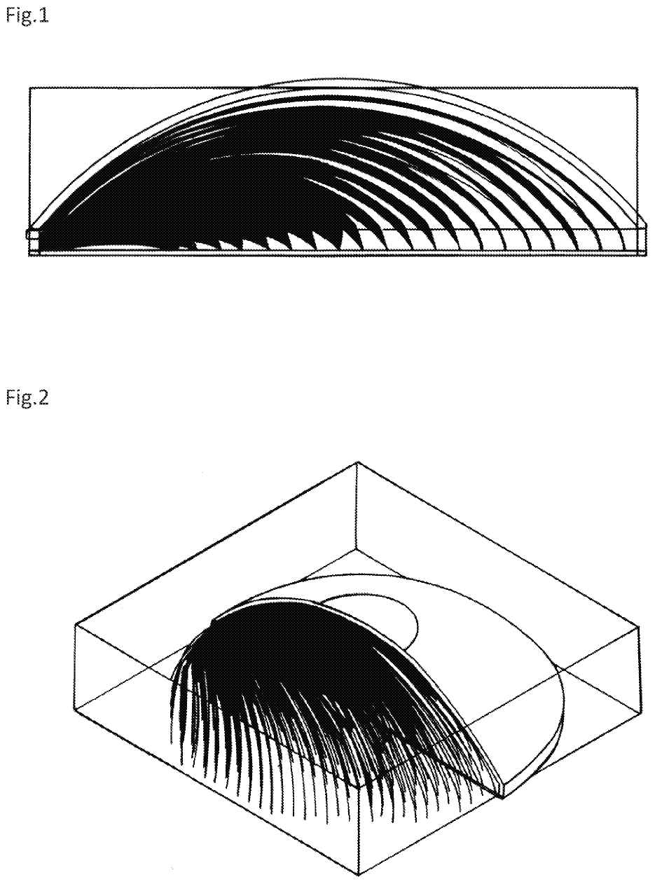

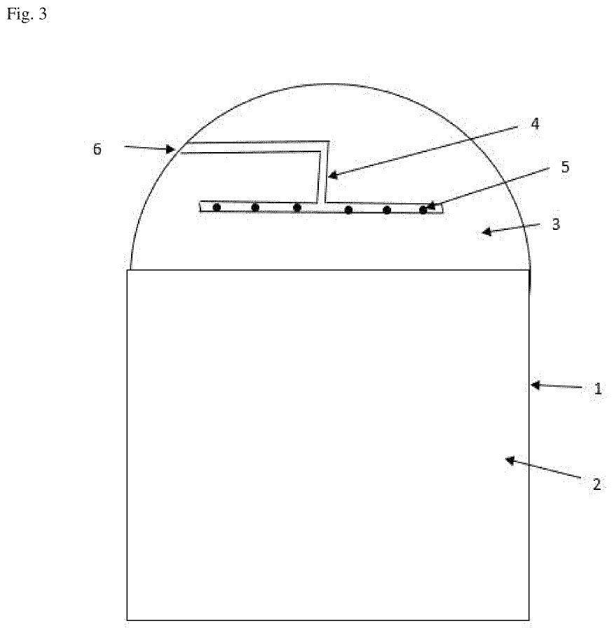

### DETAILED DESCRIPTION OF PREFERRED EMBODIMENTS

Depending on the case, the plant according to the invention may have one or more of the features below:
the micro-injectors are distributed at regular intervals along the horizontal bar of the tubular means.
the horizontal bar has a length of between 0.5 times the diameter of the chamber and 0.9 times the diameter of the chamber.
the horizontal bar comprises at least 4 micro-injectors, preferably at least 6, more preferentially still at least 8 micro-injectors.
at least one micro-injector is located less than two metres from the axis of symmetry of the chamber.
the tubular means is located less than one metre from the gas space-biomass interface.
the plant comprises a desulfurization net placed horizontally and fastened in the upper portion of the chamber and the tubular means is placed on said net.
the plant comprises a desulfurization net placed horizontally and fixed in the upper portion of the chamber and the tubular means is suspended from this net.
the tubular means is composed of a material which is resistant to a humid and corrosive atmosphere.

The plant according to the invention makes it possible to more homogeneously distribute the injected doses of the oxidizing gas within the whole of the digester. The reactions will therefore no longer be localized, which makes it possible to better reduce the hydrogen sulfide in its entirety.

In addition, this more homogeneous distribution of the oxidizing gas within the gas space of the digester enables an optimization of the amount of oxygen used and makes it possible to reduce the consumption thereof. In the case of injecting air or enriched air, the amount of nitrogen in the biogas will thus be minimal.

A further subject of the present invention is a process for producing at least partially desulfurized biogas, using a plant according to the invention, comprising:
injecting biomass into the digester;
injecting an oxidizing gas into the gas space of the digester via the system for injecting oxidizing gas; and
mixing the biomass.

The injection rate of the oxidizing gas will preferably be between 0.3% and 3% of the volume of biogas produced.

Note that the oxidizing gas might be oxygen or air or enriched air. Enriched air refers to air having a higher oxygen content than the oxygen content normally present in air.

The solution according to the invention makes it possible to obtain a biogas stream comprising less than 200 ppm of hydrogen sulfide.

The invention makes it possible to reduce the costs of purifying biogas by removal of hydrogen sulfide effectively and without any need for complex engineering.

While the invention has been described in conjunction with specific embodiments thereof, it is evident that many alternatives, modifications, and variations will be apparent to those skilled in the art in light of the foregoing description. Accordingly, it is intended to embrace all such alternatives, modifications, and variations as fall within the spirit and broad scope of the appended claims. The present invention may suitably comprise, consist or consist essentially of the elements disclosed and may be practiced in the absence of an element not disclosed. Furthermore, if there is language referring to order, such as first and second, it should be understood in an exemplary sense and not in a limiting sense. For example, it can be recognized by those skilled in the art that certain steps can be combined into a single step.

The singular forms “a”, “an” and “the” include plural referents, unless the context clearly dictates otherwise.

“Comprising” in a claim is an open transitional term which means the subsequently identified claim elements are a nonexclusive listing i.e. anything else may be additionally included and remain within the scope of “comprising.” “Comprising” is defined herein as necessarily encompassing the more limited transitional terms “consisting essentially of” and “consisting of”; “comprising” may therefore be replaced by “consisting essentially of” or “consisting of” and remain within the expressly defined scope of “comprising”.

“Providing” in a claim is defined to mean furnishing, supplying, making available, or preparing something. The step may be performed by any actor in the absence of express language in the claim to the contrary.

Optional or optionally means that the subsequently described event or circumstances may or may not occur. The description includes instances where the event or circumstance occurs and instances where it does not occur.

Ranges may be expressed herein as from about one particular value, and/or to about another particular value. When such a range is expressed, it is to be understood that another embodiment is from the one particular value and/or to the other particular value, along with all combinations within said range.

All references identified herein are each hereby incorporated by reference into this application in their entireties, as well as for the specific information for which each is cited.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

While embodiments of this invention have been shown and described, modifications thereof may be made by one skilled in the art without departing from the spirit or teaching of this invention. The embodiments described herein are exemplary only and not limiting. Many variations and modifications of the composition and method are possible and within the scope of the invention. Accordingly the scope of protection is not limited to the embodiments described herein, but is only limited by the claims which follow, the scope of which shall include all equivalents of the subject matter of the claims.

What is claimed is:

1. A plant for producing at least partially desulfurized biogas, comprising a biomass digester and/or post-digester, the digester and/or post-digester comprising:
a chamber (1) comprising the biomass (2) and the gas space (3), and
a system (6) for injecting an oxidizing gas into the gas space,
wherein the injection system comprises a tubular means (4) comprising a T-shaped portion the vertical bar of which is placed in the axis of symmetry of the chamber and the horizontal bar of which has micro-injectors (5).

2. The plant according to claim 1, wherein the micro-injectors are distributed at regular intervals along the horizontal bar of the tubular means.

3. The plant according to claim 1, wherein the horizontal bar has a length of between 0.5 times the diameter of the chamber and 0.9 times the diameter of the chamber.

4. The plant according to claim 1, wherein the horizontal bar comprises at least 4 micro-injectors, preferably at least 6, more preferentially still at least 8 micro-injectors.

5. The plant according to claim 1, wherein at least one micro-injector is located less than two metres from the axis of symmetry of the chamber.

6. The plant according to claim 1, wherein the tubular means is located less than one metre from the gas space-biomass interface.

7. The plant according to claim 1, further comprising a desulfurization net placed horizontally and fastened in the upper portion of the chamber and the tubular means is placed on said net.

8. The plant according to claim 1, further comprising a desulfurization net placed horizontally and fastened in the upper portion of the chamber and the tubular means is suspended from this net.

9. The plant according to claim 1, wherein the tubular means is composed of a material which is resistant to a humid and corrosive atmosphere.

10. A process for producing at least partially desulfurized biogas, using a plant according to claim 1, comprising:
injecting biomass into the digester;
injecting an oxidizing gas into the gas space of the digester via the system for injecting oxidizing gas; and
mixing the biomass.

11. The process according to claim 10, wherein the oxidizing gas is oxygen or air or enriched air.
